(19) United States
(12) Patent Application Publication
(10) Pub. No.: US 2021/0315603 A1
(43) Pub. Date: Oct. 14, 2021

(54) Collagen fiber extraction tool

(71) Applicant: Yan ZHENG, Chengdu (CN)

(72) Inventor: Yan ZHENG, Chengdu (CN)

(21) Appl. No.: 16/475,129

(22) PCT Filed: Mar. 14, 2019
(86) PCT No.: PCT/CN2019/078122

§ 371 Date: Jul. 1, 2019

(30) Foreign Application Priority Data
Feb. 21, 2019 (CN) 201910130710.9

(51) Int. Cl. A61B 17/32

(52) U.S. Cl. CPC A61B 17/32; A61B 2017/00792

(57) ABSTRACT

A collagen fiber extraction tool, which includes a handle, a connecting rod fixed to a front end of the handle, and a tooth member fixed to a front end of the connecting rod. The tooth member includes a body and comb teeth, the comb teeth are strip-shaped, one end of each comb tooth is fixed to the body, a tooth tip is provided at the other end of the comb tooth, and a tooth edge having an acute angle is disposed on a rear side or on both front and rear sides of the comb tooth. The plurality of comb teeth are arranged in a row along the front-rear direction of the body, and a gap is disposed between adjacent comb teeth in the same row.

### CROSS REFERENCE TO THE RELATED APPLICATIONS

This application is the national phase entry of International Application No. PCT/CN2019/078122, filed on May 14, 2019, which is based upon and claims priority to Chinese Patent Application No. 201910130710.9, filed on Feb. 21, 2019, the entire contents of which are incorporated herein by reference.

### TECHNICAL FIELD

The present disclosure relates to the technical field of plastic equipment, and in particular to a collagen fiber extraction tool.

### BACKGROUND

With the increase in amount of rhinoplasty and chin augmentation surgeries year by year, more and more filling materials used for rhinoplasty and chin augmentation surgeries are emerging. Solid synthetic materials such as silicone rubber and expanded polytetrafluoroethylene are prone to causing complications such as rejection and postoperative infection; soft synthetic fillers such as collagen and hyaluronic acid are absorbable materials, which require multiple operations for repeated filling, and the cost is higher; the transplantation of autologous tissues such as dermal flap, autologous ear cartilage, and costal cartilage leads to secondary trauma and deformity in the donor site. Acting as soft tissue filler, autologous fat granules are becoming more and more popular with plastic surgeons because of the advantages including rich sources, easily being get, simple operation, resulting in good filling shape and leading to no rejection reaction. However, the autologous fat granules also have a certain absorption rate (which is lower than that of collagen, hyaluronic acid, etc.) after being transplanted, and in the nasal back, chin, etc., the survival rate of autologous fat granules after transplantation is very low due to factors such as the small amount of local tissue, the non-rich vascular network, and the large skin tension, etc., thus making it difficult to achieve a desired effect.

The patent (application No. CN201811283679.4) of the present inventor relates to a kind of fat transplantation material (also called lipid collagen), which is suitable for rhinoplasty and chin augmentation surgeries and the like because of its characteristics including high density, high viscosity and strong supporting force, etc. At present, in extracting lipid collagen, a negative pressure swelling liposuction method is used to obtain granular fat, and lipid collagen is extracted from the suctioned granular fat. On average, 1 ml of lipid collagen can be extracted from each 7-10 ml of the granular fat, and the content of collagen fibers in the lipid collagen is about 30%-60%. The collagen fibers are ideal fillers. The extraction rate of extracting lipid collagen by this method is low, and a large amount of granular fat needs to be suctioned, which is harmful to the human body. Moreover, the content of collagen fibers in the extracted lipid collagen is insufficient and the content is unstable.

### SUMMARY

An object of the present disclosure is to provide a collagen fiber extraction tool for solving the problem in the prior art of low extraction rate of lipid collagen, insufficient content of collagen fibers in the lipid collagen and unstable content when a negative pressure swelling liposuction method is used to obtain granular fat and lipid collagen is extracted from the suctioned granular fat.

In order to achieve the above object, embodiments of the present disclosure provide the following technical solutions:

A collagen fiber extraction tool, which includes a handle, a connecting rod fixed to a front end of the handle, and a tooth member fixed to a front end of the connecting rod, wherein the tooth member includes a body and comb teeth for winding collagen fibers; the comb teeth are strip-shaped, one end of each comb tooth is fixed to the body, a tooth tip is provided at the other end of the comb tooth, and a tooth edge for cutting collagen fibers is disposed on a rear side or on both front and rear sides of the comb tooth.

The comb teeth are provided in plurality, and the plurality of comb teeth are arranged in a row along the front-rear direction of the body, and a gap is disposed between adjacent comb teeth in the same row.

The comb teeth are provided in one or more rows, and the plurality rows of comb teeth are evenly distributed in a circumferential direction of the body.

The embodiment of the present disclosure is further characterized in that the comb teeth of the same row are perpendicularly fixed to the body.

The embodiment of the present disclosure is further characterized in that ends of the comb teeth of the same row that are provided with the tooth tips are inclined forward or backward.

The embodiment of the present disclosure is further characterized in that the body is integrally formed with the connecting rod.

The embodiment of the present disclosure is further characterized in that the comb teeth have a height of 1-10 mm and a tooth width of 0.5-2 mm, and the tooth spacing between adjacent comb teeth of the same row is 0.1-5 mm.

The embodiment of the present disclosure is further characterized in that the comb teeth have a height of 2-3 mm and a tooth width of 1-1.5 mm, and the tooth spacing between adjacent comb teeth of the same row is 1-2 mm.

The embodiment of the present disclosure is further characterized in that the length of each row of comb teeth is 1-20 cm.

The embodiment of the present disclosure is further characterized in that the collagen fiber extraction tool further includes a handle which is cylindrical, a front end of the handle is connected to a rear end of the connecting rod, and a groove is disposed on the handle.

The embodiment of the present disclosure is further characterized in that the heights of the comb teeth of each row are decreased towards both front and rear ends from the middle.

The embodiment of the present disclosure is further characterized in that the heights of the comb teeth at the front end of each row are successively decreased forward, and the heights of the comb teeth at the rear end of each row are successively decreased rearward.

The present disclosure has the following advantages: when the collagen fiber extraction tool provided by the present disclosure rotates and reciprocates in the fat tissue injected with the swelling solution, the collagen fibers can be wound around the comb teeth, and stored in the gaps between the comb teeth. When the collagen fiber extraction tool is taken out, an extract containing a large amount of collagen fibers is obtained with no fat being suctioned, thereby reducing the damage to the human body. More than 90% of the extract is collagen fibers, and the remaining substance is granular fat or other substance. The content of collagen fibers is high and the content is stable. In addition, the collagen fiber extraction tool provided by the embodiment has the advantages of simple structure and elaborate design, and is suitable for production and use. Moreover, the extracted collagen fibers are coarse, have a low absorption rate when used as filler, and have a strong supporting force and a good plastic effect, thus making it suitable for rhinoplasty and chin augmentation surgeries and for filling supraorbital ridge, tear trough, nasolabial fold, lip, etc.

### BRIEF DESCRIPTION OF THE DRAWINGS

FIG. 1 is a schematic structural view of a collagen fiber extraction tool according to a first embodiment of the present disclosure;

FIG. 2 is a schematic structural view of another collagen fiber extraction tool according to the first embodiment of the present disclosure;

FIG. 3 is a cross-sectional view taken along line A-A of FIG. 1;

FIG. 4 is a schematic structural view of a collagen fiber extraction tool according to a second embodiment of the present disclosure.

In the drawings: 1—handle, 11—groove, 2—connecting rod, 3—tooth member, 31—body, 32—comb teeth, 321—tooth tip, 322—tooth edge.

### DETAILED DESCRIPTION OF THE EMBODIMENTS

The following embodiments are used to illustrate the present disclosure, but are not intended to limit the scope of the present disclosure.

### First Embodiment

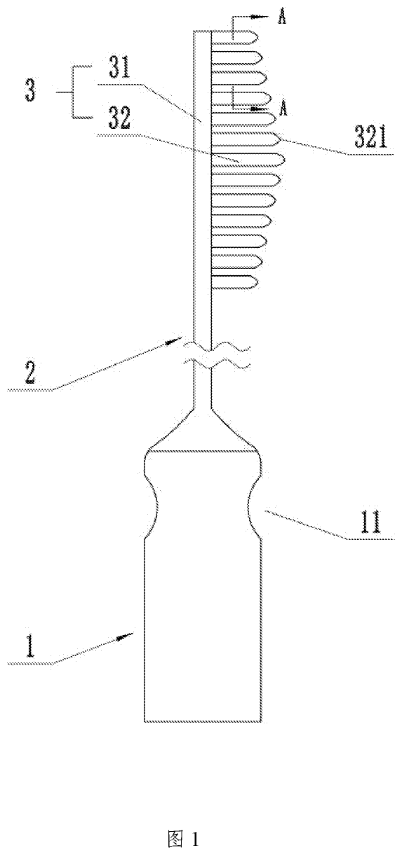

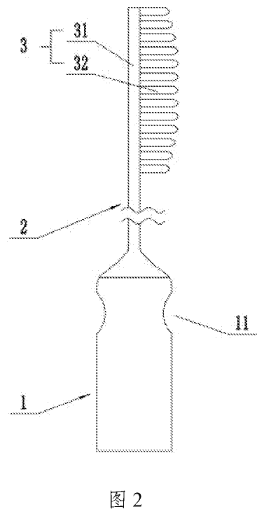

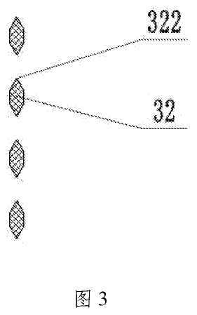

As shown in FIGS. 1, 2 and 3, the first embodiment provides a collagen fiber extraction tool, which includes a handle 1, a connecting rod 2 fixed to a front end of the handle 1, and a tooth member 3 fixed to a front end of the connecting rod 2, wherein the handle 1 is cylindrical, and a groove 11 is disposed on the handle 1 for gripping. The tooth member 3 includes a body 31 and comb teeth 32, wherein the body 31 is integrally formed with the connecting rod 2, one end of each comb tooth 32 is fixed to the body 31, a sharp tooth tip 321 is provided at the other end of the comb tooth 32, and the comb teeth 32 are used to wind the collagen fibers in the extracting process. The comb teeth 32 are strip-shaped and are provided in plurality, and the plurality of comb teeth 32 are arranged in a row. The length of a row of comb teeth 32 is provided along the front-rear direction of the body 31. A gap is disposed between adjacent comb teeth 32 in a row of comb teeth 32. The heights of the comb teeth 32 of a row of comb teeth 32 are decreased towards both front and rear ends from the middle of the row. When viewed from the side, the tooth tips 321 of a row of comb teeth 32 are arranged in an arc shape; alternatively, the heights of the comb teeth 32 at the front end of a row of comb teeth 32 are successively decreased forward, and the heights of the comb teeth 32 at the rear end are successively decreased rearward. When viewed from the side, the tooth tips 321 of a row of comb teeth 32 are flush in the middle, and are declined at an acute angle or in an arc shape at both ends. The same fixed angle may be set between the comb teeth 32 of the same row and the body 31. For example, the comb teeth 32 of the same row may be perpendicularly fixed to the body 31, or ends of the comb teeth 32 of the same row that are provided with the tooth tip 321 are inclined forward, or ends of the comb teeth 32 of the same row that are provided with the tooth tip 321 are inclined backward. That is, in the latter two cases, there is a certain inclined angle between the comb teeth 32 of the same row and the body 31. Tooth edges 322 having an acute angle are disposed on both front and rear sides of the comb teeth 32. The acute angle of the tooth edge 322 is 15°-45°, and the tooth edge 322 is configured to cut the collagen fibers in the extracting process.

In use, a swelling solution is first injected in a fat-rich body part such as the thigh or abdomen, then an incision is made in a secluded body part, and subsequently the collagen fiber extraction tool is inserted into deep fat tissues. With the rotation of the handle 1, the collagen fibers in the fat tissues will be wound onto the comb teeth 32. The tool reciprocates at the same time of rotating, or after several turns of rotation, the insertion depth is adjusted so that the collagen fibers of new fat tissues can be extracted. After a certain number of turns of rotation, the collagen fiber extraction tool is taken out (the number of turns of rotation can be determined as required). Since the heights of the comb teeth 32 of a row of comb teeth 32 are decreased towards both front and rear ends from the middle of the row, the smoothness can be effectively ensured while the tool is inserted and taken out. During the rotation process, the gaps between adjacent comb teeth 32 can accommodate more collagen fibers, and more collagen fibers can be obtained by one extraction, resulting in a high extraction efficiency; during the reciprocating and shifting process, the tooth edge 322 can cut out the collagen fibers wound on the comb teeth 32 from the collagen fibers in the fat tissues, thereby avoiding the collagen fibers being forcedly pulled and dragged during the shifting, and reducing the damage to the collagen fibers retained in the fat tissues. After the collagen fiber extraction tool is taken out, the collagen fibers are removed with scissors and forceps. The extraction is performed again, and repeated until the amount of collagen fibers obtained can satisfy the filling requirement. It is not necessary to suction fat, and the damage to the human body is reduced. It can be ensured that the extraction site does not deform, and it is not necessary to wear a corset after surgery. During the surgery, two collagen fiber extraction tools are generally used at the same time, so that when the fibers wound on one collagen fiber extraction tool are torn off, another collagen fiber extraction tool can continue the operation of extracting collagen, thus saving time. A scraping comb is further provided for use in combination with the tool. The scraping comb has a tooth spacing that is equal to the tooth spacing of the comb teeth 32 of the collagen fiber extraction tool to scrape the wound collagen fibers from the comb teeth 32.

The collagen fiber extraction tool provided by the embodiment has a simple structure and an elaborate design, and is suitable for production and use. The extracted collagen fibers have a high purity, and the content of collagen fibers can be up to more than 90%. Even if a certain amount of granular fat is contained, the content of collagen fibers in the extract is extremely stable. Moreover, the extracted collagen fibers are coarse, have a low absorption rate when used as filler, and have a strong supporting force and a good plastic effect, thus making it suitable for rhinoplasty and chin augmentation surgeries and for filling supraorbital ridge, tear trough, nasolabial fold, lip, etc.

In the present embodiment, the comb teeth 32 have a height of 1-10 mm and a tooth width of 0.5-2 mm, the tooth spacing between adjacent comb teeth 32 of the same row is 0.1-5 mm, and the length of each row of comb teeth 32 is 1-20 cm, thus making the tool suitable for making a minimally invasive incision. In a preferred solution of the present embodiment, the comb teeth 32 have a height of 2-3 mm and a tooth width of 1-1.5 mm, and the tooth spacing between adjacent comb teeth 32 of the same row is 1-2 mm. The preferred sizes not only ensures the extraction efficiency, but also ensures a small incision size, thus reducing damage to the human body.

In a preferred solution of the present embodiment, the handle 1 can be manufacture as an electric handle 1, such as the handle 1 of a small electric drill used in dentistry. By using the electric handle 1, not only physical labor is saved, but also rotational speeds of the connecting rod. 2 and the tooth member 3 are effectively increased, thereby further improving the extraction efficiency.

### Second Embodiment

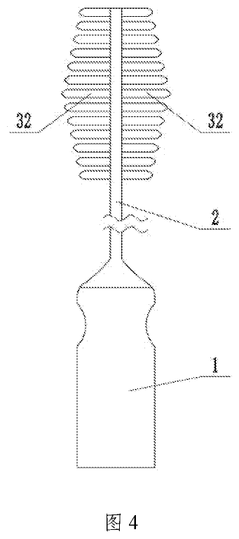

As shown in FIG. 4, the second embodiment provides another collagen fiber extraction tool, which has substantially the same structure as that of the first embodiment, except that the tooth member 3 includes a body 31 and two rows of comb teeth 32, wherein the two rows of comb teeth 32 are distributed 180° apart in the circumferential direction of the body 31. In use, the collagen fiber extraction tool provided by the second embodiment makes an incision on the human body that is slightly larger than that of the first embodiment, but the extraction efficiency can be doubled.

### Third Embodiment

The third embodiment provides another collagen fiber extraction tool, which has substantially the same structure as that of the first embodiment, except that the tooth member 3 includes a body 31 and three rows of comb teeth 32, wherein the three rows of comb teeth 32 are distributed 120° apart in the circumferential direction of the body 31. As compared to the collagen fiber extraction tool provided by the first embodiment, the extraction efficiency is tripled in the collagen fiber extraction tool provided by the third embodiment.

While the present disclosure is described in detail with reference to the specific embodiments and general description above, it is apparent to those skilled in the art that some modifications or improvements may be made on the basis of the present disclosure. Therefore, such modifications or improvements made without departing from the spirit of the disclosure will all fall within the scope of protection of the disclosure.

1. A collagen fiber extraction tool, comprising: a handle, a connecting rod fixed to a front end of the handle, and a tooth member fixed to a front end of the connecting rod; wherein the tooth member comprises a body and a plurality of comb teeth for winding collagen fibers; the plurality of comb teeth are strip-shaped, one end of each comb tooth is fixed to the body, a tooth tip is provided at an other end of the each comb tooth, and a tooth edge for cutting the collagen fibers is disposed on a rear side or on both front and rear sides of the each comb tooth;
wherein the plurality of comb teeth are arranged in a row along a front-rear direction of the body, and a gap is disposed between adjacent comb teeth in a same row; and
wherein the plurality of comb teeth are provided in one or more rows, and the one or more rows of comb teeth are evenly distributed in a circumferential direction of the body.

2. The collagen fiber extraction tool according to claim 1, wherein the plurality of comb teeth of the same row are perpendicularly fixed to the body.

3. The collagen fiber extraction tool according to claim 1, wherein ends of the plurality of comb teeth of the same row provided with the tooth tip are inclined forward or backward.

4. The collagen fiber extraction tool according to claim 1, wherein the body is integrally formed with the connecting rod.

5. The collagen fiber extraction tool according to claim 1, wherein the plurality of comb teeth have a height of 1-10 mm and a tooth width of 0.5-2 mm, and the tooth spacing between adjacent comb teeth of the same row is 0.1-5 mm.

6. The collagen fiber extraction tool according to claim 5, wherein the plurality of comb teeth have a height of 2-3 mm and a tooth width of 1-1.5 mm, and the tooth spacing between adjacent comb teeth of the same row is 1-2 mm.

7. The collagen fiber extraction tool according to claim 6, wherein a length of each row of comb teeth is 1-20 cm.

8. The collagen fiber extraction tool according to claim 1, wherein a groove is disposed on the handle.

9. The collagen fiber extraction tool according to claim 1, wherein heights of the plurality of comb teeth of each row decrease towards both front and rear ends from the middle.

10. The collagen fiber extraction tool according to claim 1, wherein heights of the plurality of comb teeth at the front end of each row successively decrease forward, and heights of the plurality of comb teeth at the rear end of each row successively decrease rearward.
